(19) United States
(12) Patent Application Publication
(10) Pub. No.: US 2014/0308655 A1
(43) Pub. Date: Oct. 16, 2014

(54) Device for Manipulating Biological Materials in a Process of Cryopreservation and a Use of Such a Device

(71) Applicants: Teresa Mogas, Cerdanyola de Valles (ES); Roser Morato, Cerdanyola de Valles (ES); Andreu Fontova, Cerdanyola de Valles (ES)

(72) Inventors: Teresa Mogas, Cerdanyola de Valles (ES); Roser Morato, Cerdanyola de Valles (ES); Andreu Fontova, Cerdanyola de Valles (ES)

(73) Assignee: UNIVERSITAT AUTONOMA DE BARCELONA, Bellaterra (Barcelona) (ES)

(21) Appl. No.: 13/699,038

(22) PCT Filed: May 20, 2011
(86) PCT No.: PCT/EP11/02529

§ 371 Date: Mar. 12, 2013

(30) Foreign Application Priority Data
May 21, 2010 (EP) 10380072.8

(51) Int. Cl. C12N 5/076

(52) U.S. Cl. CPC C12N 5/061
USPC 435/2; 435/307.1

(57) ABSTRACT

The device comprises a loading portion (2a) for loading biological material thereon, and a channel (C) arranged for conducting a warming solution there through and for applying it over said biological material while loaded on said loading portion (2a), for an in-situ warming. The use comprises simultaneously warming and transferring a cryopreserved biological material loaded on a device loading portion, by conducting and applying a warming solution on the biological material loaded on the device loading portion.

### FIELD OF THE ART

The present invention generally relates, in a first aspect, to a device for manipulating biological materials in a process of cryopreservation, and more particularly to a device comprising a channel for applying a warming solution over a biological material while loaded on said device, for an in-situ warming.

A second aspect of the invention relates to a use of a device for manipulating biological materials in a process of cryopreservation comprising simultaneously warming and transferring a cryopreserved biological material.

### PRIOR STATE OF THE ART

Cryopreservation of biological materials, particularly of embryos, is a crucial step for the widespread and conservation of animal genetic resources. The development of effective methods of freezing embryos by the slow freezing techniques has made embryo transfer a much more efficient technology, which no longer depends on the immediate availability of suitable recipients as for fresh embryo transfer. In the bovine species, freezing embryos is now common and pregnancy rates are only slightly less than those achieved with fresh embryos (Leibo and Mapletoft 1998). In this approach, a 0.25 mL plastic straws are the most usual containers to slow freeze embryos because thawing of the embryos is done directly in a water bath, much like semen, and its content is deposited directly into the uterus of the recipient female, as occurs in artificial insemination (AI). There is no need for a microscope or complicated dilution procedures. The cryoprotectant leaves the embryo in the uterus, without causing osmotic stress. The level of skill required to transfer these embryos is the same as that needed for conventionally AI and no embryologist is required at the time of thawing. Consequently, a growing number of direct-transfer embryos are now being transferred by technicians with experience in AI. However slow freezing procedures are time-consuming and require the use of biological freezers. For this reason, complicated embryo freezing procedures are being replaced by a relatively simple procedure called vitrification.

Vitrification has become a viable and promising alternative to traditional approaches because does not require expensive programmable cell freezers used for slow freezing procedures and the equilibration and subsequent cryopreservation are easier and takes less time for embryos. The physical definition of vitrification is the solidification of a solution at a temperature below the glass transition temperature of the solution, not by ice crystallization, but by extreme elevation in viscosity, using high cooling rates from 15,000 to 30,000° C. per minute (Martino et al. 1996; Vajta et al. 1998). However, there are two main shortcomings to the vitrification technique: [1] high concentrations of cryoprotectants are required, which may be toxic to oocytes and embryos, and [2] if cooling rate is insufficiently fast, intracellular ice and chilling injury may occur. To overcome these problems, several methods have been devised in which very small sample volumes are used. For example, decreasing the sample volume to 0.1-1 μL and the use of carrier systems of minimum capacity increase the rate at which heat is conducted out of the sample, and the sample therefore cools at a very fast rate. A variety of these techniques can be found in the literature (Chen et al. 2001; Isachenko et al. 2003b; Lane et al. 1999; Liebermann et al. 2002; Martino et al. 1996; Matsumoto et al. 2001; Vajta et al. 1998) and have been applied with varying degrees of success in several mammalian species, including humans.

Stem cells have been used in a clinical setting for many years. Haematopoietic stem cells have been used for the treatment of both haematological and non-haematological disease; while more recently mesenchymal stem cells derived from bone marrow have been the subject of both laboratory and early clinical studies. Whilst these cells show both multipotency and expansion potential, they nonetheless do not form stable cell lines in culture which is likely to limit the breadth of their application in the field of regenerative medicine. Human embryonic stem cells are pluripotent cells, capable of forming stable cell lines which retain the capacity to differentiate into cells from all three germ layers. This makes them of special significance not only for research but also for clinical applications as an unlimited source of cells for transplantation and tissue regeneration (Wobus et al., 2001). Induced pluripotent stem (iPS) cells may also provide a similar breadth of utility without some of the confounding ethical issues surrounding embryonic stem cells. An essential pre-requisite to the commercial and clinical application of stem cells are suitable cryopreservation protocols for long-term storage.

Whilst effective methods for cryopreservation and storage have been developed for haematopoietic and mesenchymal stem cells, embryonic cells and iPS cells have proved more refractory. Vitrification technology presents new opportunities for preservation of isolated embryo-derived stem cells without first establishing a viable embryonic stem cell line offering a new avenue for banking the stem cell source material. Vitrification protocols have also been successfully used for cryopreservation of human embryonic stem cell lines (Richards et al., 2004; Li et al., 2010). On the other hand, as stated above, vitrification methods have become more and more popular in recent years, mainly because of the higher survival rate compared to that with slow freezing, especially for cells that are sensitive to cryoinjury (Abdelhafez et al., 2010).

The latest approach to minimum volume vitrification is the cryotop device (Kitazato Supply Co., Fujinomiya, Japan) designed by Dr. M. Kuwayama. The system consists of a fine, transparent polypropylene film attached to a plastic handle and equipped with a cover straw, in which oocytes or embryos can be loaded in a very small volume (<0.1 μL) (Kuwayama et al. 2005). Cryotops have been successfully used to cryopreserve human oocytes and embryos (Antinori et al. 2007; Cobo et al. 2007; Lucena et al. 2006; Stehlik et al. 2005), immature and in vitro-matured horse oocytes (Bogliolo et al. 2006), cow, sheep and buffalo oocytes (Chian et al. 2004; Gasparrini et al. 2007; Succu et al. 2007), rabbit zygotes (Hochi et al. 2004), in vitro-produced pig embryos (Ushijima et al. 2004), porcine blastocysts produced by parthenogenetic activation or somatic cell nuclear transfer from delipidated in vitro-matured oocytes (Du et al. 2007), cow and buffalo embryos (Laowtammathron et al. 2005), as well as the oocytes of exotic species such as the minke whale (Iwayama et al. 2004).

WO02085110A1, by KABUSHIKIKAISHA KITAZATO SUPPL. et al, discloses one of said cryotop devices, describing particularly an egg freezing preservation tool comprising a main body formed of cold-resistant material, an egg adherence holding strip attached to one end of the main body and formed of flexible, transparent and liquid nitrogen-resistant material, and a cylindrical member closed at one end, where the egg adherence holding strip is removably and packageably attached to the main body and formed of cold-resistant material.

Another of said cryotop devices is disclosed in EP1619243A1, by KUWAYAMA MASASHIGE et al, which cryotop device is an improvement of the one disclosed in WO02085110A1, and is in the form of an egg freezing and storing instrument which has an egg freezing and storing tube made of a liquid nitrogen-resistant material and a metal cylindrical protection member for protecting the tube. The tube has a body part and an egg-storing small-diameter part having an inner diameter of 0.1 mm to 0.5 mm. The tube can be heat-sealed at a front side of the small-diameter part and at the body part. The cylindrical protection member has a tubular part for accommodating a front side of the small-diameter part of the tube, and a semi-tubular part for accommodating a portion of the small-diameter part not accommodated in the tubular part and a front portion of the body part.

Embryos cryopreserved by vitrification must be warmed by placing them in a hypertonic solution to remove the permeating cryoprotectants before transferring them to an isotonic culture medium. Many authors report the use of one or more dilution steps with different concentrations of sucrose, which controls the degree of swelling during cryoprotectant removal (Isachenko et al. 1997; Kobayashi et al. 1990; Szell and Shelton 1986). The introduction of the vitrification technology into current practice would require that embryos vitrified could be warmed in-straw dilution in order to be directly transferred to the uterus, at the same level of difficulty as one AI. This in-straw dilution and direct transfer method reduces the requirement for equipment and technical skill which are obstacles to application of embryo transfer technology at many farms where appropriate conditions are not available. Until relatively recently, standard 0.25 mL insemination straws were used for vitrifying and field warming of embryos. However, pregnancy rates were lower than the ones obtained by the slow freezing techniques.

Several types of embryo containers have been developed as substitutes of the semen straws to both reduce the volume of vitrification solution surrounding embryos and to facilitate the process of cryopreservation by reducing the time required for the procedure. These novel containers increase the cooling rate during vitrification without ice crystal formation after immersion in the liquid nitrogen. Kuwayama et al. (2005) reported that the use of a cryotop container improved the survival rate of thawed embryos and oocytes.

During the warming process, most of the vitrification devices as nylon mesh, metal mesh, cryoloop, cryotip and cryotop need the use of a dissecting microscope in order to plunge the embryos in the initial warming solution. Few reports described the thawing of vitrified embryos in field conditions. Isachenko et al. (2003a) reported a fast microscope-free thawing of ovine embryos and direct transfer using the open-pulled straw as a device. However, it is well documented that survival results after embryo vitrification using the OPS system are poorer than those obtained using the cryotop method.

Next some patents documents are cited disclosing different devices for manipulating biological materials in a process of cryopreservation, said manipulation including a warming stage of the biological materials.

WO2006/066385 discloses a tool for manipulating a sample of developmental cells, like oocytes or embryos, in a process of cryopreservation. The tool includes an elongated stem with a sample loading portion connected at one end and a manipulation portion at the other end. The tool also includes a sleeve which is telescopically mounted onto the stem, with the manipulation portion extending from the sleeve out a first open end thereof and the loading portion being extendable from the sleeve out a second open end. The sample loaded onto the loading portion can be easily retracted within the sleeve for protection by a user pulling the manipulation portion while maintaining the sleeve. The warming of the vitrified development cell is carried out by the immersion of the loading portion with the sample into a thawing solution.

U.S. Pat. No. 5,160,312 concerns to a cryoprotector tubular container which allows the direct transfer of embryos. The container is used for freezing an embryo contained therein and also for unfreezing said embryo, for which purpose the container defines two chambers separated by an air bubble: one chamber for containing the embryo and another chamber for containing a warming solution. By shaking the container the two chambers get communicated, thus the unfreezing of the embryo is achieved in order to be in field transferred.

WO0192478A1 discloses a straw for vitrifying and transferring embryos, the warming of the vitrified embryo being carried out by the immersion of the straw portion containing the embryo in a bain-marie.

WO2009064708A2 relates to a cryocontainer for vitrifying an embryo, which is made of materials with shape memory, which varies its characteristics as a function of heat, in order to pass from a state suitable for managing the embryo to a state suitable for transferring heat in a fast manner. The warming of the embryo is only described to be carried out by means of its immersion into a warm water bath.

US2008220507A1 discloses an assembly for packaging a volume of substance to be preserved by cryogenic vitrification, which comprises a sheathing including a thin tube having a predetermined internal diameter, which is preferably between 0.95 and 2.55 mm, and a predetermined length and a support comprising a zone for receiving said predetermined substance volume, said support capable of being introduced inside said thin tube through a mouth thereof.

Said sheathing is closed at an end opposite said mouth, by welding, and a second welding is realised near said mouth once the support with the substance is inside the sheathing, in order to be immersed vertically, to facilitate storage, in a cryogenic liquid (for example liquid nitrogen) to vitrify the substance with a view to its cryopreservation. Such a sheathing is provided for avoiding the risk of contamination of the substance, thus both of said weldings seal the sheathing from gas and liquids leakages and ingresses.

Although the support of the assembly of US2008220507A1 is a hollow tube, the use of its inner channel is only related to substance loading tasks, whether to be partially occupied by the substance to vitrify or, for the embodiment shown in its FIG. 6, for aspirating the substance from a loading end of the tube, by applying a vacuum supply to the opposite end of the tube inner channel. Those substance loading tasks are always performed before introducing the support into the sheathing.

No other use for the tube inner channel is disclosed in US2008220507A1.

Neither said tube inner channel nor the sheathing are arranged, dimensioned or configured for allowing using said tube inner channel while the tube is already into the sheathing.

None of the cited prior art proposals describes a device for manipulating biological materials in a process of cryopreservation which permits to apply a warming solution on the biological material loaded on the device by conducting it through a channel comprised by the device, therefore allowing warming procedure directly in field conditions without the need for more equipment.

### DESCRIPTION OF THE INVENTION

It is necessary to offer an alternative to the state of the art which covers the gaps found therein and which provides such an, until now, unknown device.

To that end, the present invention provides, in a first aspect, a device for manipulating biological materials in a process of cryopreservation, such as vitrification, comprising a loading portion for loading biological material thereon and, on contrary to the known devices for manipulating biological materials, further comprising:
an elongated handle portion with a channel defined through at least part of its interior, along its length, said channel being arranged for conducting a warming solution there through and for applying it over said biological material while loaded on said loading portion, for an in-situ warming; and
a container closed at an end and having a mouth, said container being arranged, dimensioned and configured for:
   being removably coupled to said elongated handle portion by the introduction of the latter through said container mouth, covering at least said loading portion, such that, when coupled, inner air cannot escape via said mouth; and
   receiving, when coupled, both said applied warming solution and said biological material, once warmed and swept away from said loading portion by said applied warming solution, without inner air pushing said warming solution towards the container mouth by an inner air counter pressure.

For an embodiment, said container is a cover straw which is closed at said end by means of a plug, or stopper, made of a porous material permeable to gases and impermeable to liquids, such that, when said warming solution is being introduced into the container inner air can escape through said plug to allow the warming solution to occupy inner air previous place, in order not to generate said inner air counter pressure.

Said plug is, for an embodiment, also made of a second material that once wet by said warming solution do not let liquids percolate there through and thus becomes a plunger which, by sliding, can propel the container contents through said mouth when the container is uncoupled from said elongated handle portion and said plunger is pushed by external actuation means, such as the ones provided by an insemination catheter.

Said cover straw is also used for protection of said biological material loaded thereon and of the device, from mechanical damage, during storage and manipulation thereof.

For a preferred embodiment said cover straw is a semen straw, where said plug is a wick and powder plug made of two porous pads (for example made of cotton) plus a powder gellable by hydration in order to become such a plunger when said powder gelifies, and configured for also receiving both said applied warming solution and said biological material suspended there in, once warmed and swept away from said loading portion by said applied warming solution. The cover straw is preferably dimensioned to receive and hold all the volume of the applied warming solution, once the inner air has escaped through the porous plug.

The diameter of said plug, or stopper, is for semen straws greater than or equal to approximately three millimetres with a liquid while it is vertical, i.e. when the porous pads are impregnated with liquid, in this case with the warming solution.

The tube constituting said cover straw is a rectilinear length of a generally transparent tubular envelope.

Generally said cover straw is also configured for, when uncoupled from said handle portion, being mounted into an insemination catheter for direct transferring said biological material suspended in the warming solution into a final target, such as the uterus of an animal, when the biological material is an embryo, or tissue culture when the biological material are stem cells or oocytes.

For an alternative embodiment the container is a cover straw made of a porous material, such that, when said warming solution is being introduced into the container inner air can escape through the cover straw walls to allow the warming solution to occupy inner air previous place, in order not to generate said inner air counter pressure.

For another embodiment, the container has two portions with different section dimensions, a first one including said mouth and a second one with a wider section dimension, the dimensional ratio between said two portions section dimensions providing that said warming solution enters the container by dripping, settles in the second portion bottom and is not pushed towards the container mouth by inner air counter pressure, not even when retrieving said elongated handle portion.

A second aspect of the invention relates to a use of a device for manipulating biological materials in a process of cryopreservation, comprising simultaneously warming and transferring a cryopreserved biological material loaded on a device loading portion, by conducting and applying a warming solution on said biological material loaded on said device loading portion.

According to an embodiment, the use comprises performing said warming solution conduction through an inner channel of said device.

The use of the second aspect comprises, for an embodiment, performing said transfer from a handle portion of said device defining said inner channel to a container of said device.

Another embodiment of the use of the second aspect of the invention comprises performing a second transfer from said container to a final target.

The device used by the use of the second aspect of the invention is, for several embodiments, the device of the first aspect of the invention.

A methodology for using the device of the first aspect of the invention comprises:

a) loading biological material on a loading portion of the device;

b) cryopreserving said biological material loaded on said loading portion; and

c) warming said cryopreserved biological material loaded on said loading portion, said step c) being carried out by conducting and applying at least one warming solution (and if necessary a dilution solution) on said biological material loaded on said loading portion, using said device.

Referring to the biological material, depending on the embodiment is a biological material, or a sample thereof, selected from the group comprising: an oocyte, an embryo, a cell, such as a stem cell, and a tissue.

For a preferred embodiment said cryopreserving of said step b) is carried out by a vitrification technique.

Said use methodology further comprises, for an embodiment, after said step b), removably covering said loading portion of said device with a cover straw, closed at an end by, for example, a cotton plunger, and generally also covering most or the entire handle portion.

In order to carry out the referred biological material transfer, the use methodology comprises, for an embodiment, sweeping away the biological material from the loading portion towards said cover straw, by applying said warming solution with a flow which is sufficient for that purpose, and receiving, into said cover straw, both said applied warming solution and said biological material suspended there in, together with the diluted cryoprotectant agents.

The use methodology comprises, according to one embodiment, a step d) of direct transferring the warmed biological material into the uterus of an animal, by means of inserting said cover straw containing said biological material being suspended on the warming solution into an insemination catheter, by introducing there into at least part of said insemination catheter, with the closed end coupled to the catheter end, and by propelling the cover straw content towards the animal uterus by pushing the cotton plunger with the aid of said insemination catheter in a syringe-like way.

The device and use provided by the present invention are particularly suitable for embryo vitrification and provide microscope-free warming and direct embryo transfer. The advantages of this device and use are that warming is easily and rapidly achieved, by directly addition of the warming solution or solutions through a hollow handle connected to, for example, a standard syringe by a LUER connector, and embryo transfer is directly performed without the need for optical equipment and, for an embodiment, using a semen straw as the protective cover, being optimal for its use in field conditions.

### BRIEF DESCRIPTION OF THE DRAWINGS

The previous and other advantages and features will be more fully understood from the following detailed description of embodiments with reference to the attached drawings, which must be considered in an illustrative and non-limiting manner, in which:

FIG. 1 is a side view of the device of the first aspect of the invention, for an embodiment;

FIG. 2 is a plan view of the device of the invention for the same embodiment illustrated in FIG. 1;

FIG. 3 is a cross sectional view taken along the plane indicated by line III-Ill in FIG. 2;

FIG. 4 is an exploded plan view which shows an end of the handle portion of the device of the first aspect of the invention and an attachment support to be coupled thereto;

FIG. 5 is an exploded side view showing the same elements as FIG. 4;

FIGS. 6a and 6b show two attachment supports for two respective embodiments alternative to the one illustrated by FIG. 4;

FIG. 7 is a perspective view which shows the device of the first aspect of the invention for the same embodiment of FIGS. 1 to 3;

FIG. 8 shows, in a perspective view, a pipette, which is not part of the invention, with a LUER connector couplable to a mating connector of the device of the first aspect of the invention;

FIG. 9 shows, in a perspective view, the pipette of FIG. 8 with its LUER connector coupled to the connector of the device of the first aspect of the invention, for an embodiment;

FIGS. 10 and 11 are, respectively, an unexploded view and an exploded view of the device of the first aspect of the invention, for an embodiment where the device comprises a cover straw, and where a syringe, which is not part of the invention, is used in order to supply a warming solution to the inner channel of the device;

FIGS. 12a and 12b show the handle portion of the device of the first aspect of the invention and an attachment support to be coupled thereto, shown coupled in FIG. 12a and in an exploded view in FIG. 12b, by coupling means different from the ones of FIGS. 4 and 5, for an alternative embodiment;

FIG. 13 shows the device of the first aspect of the invention for an embodiment where it comprises a semen straw, by means of a sequence of four images, a, b, c and d, representing corresponding successive stages of its use for transferring the biological material together with the warming solution into the semen straw; and

FIG. 14 shows a sequence of four images, a, b, c and d, representing corresponding successive stages of the use of the device for transferring the biological material together with the warming solution into a container having portions with different section dimensions, for another embodiment of the device of the first aspect of the invention.

### DETAILED DESCRIPTION OF SEVERAL EMBODIMENTS

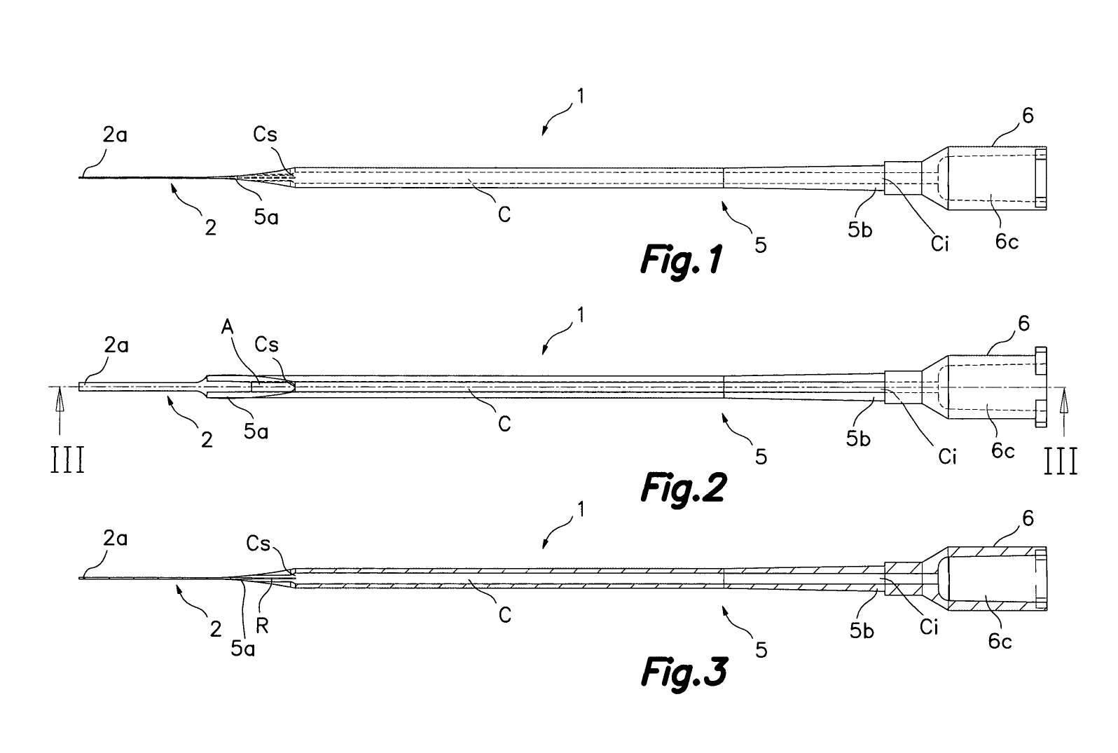

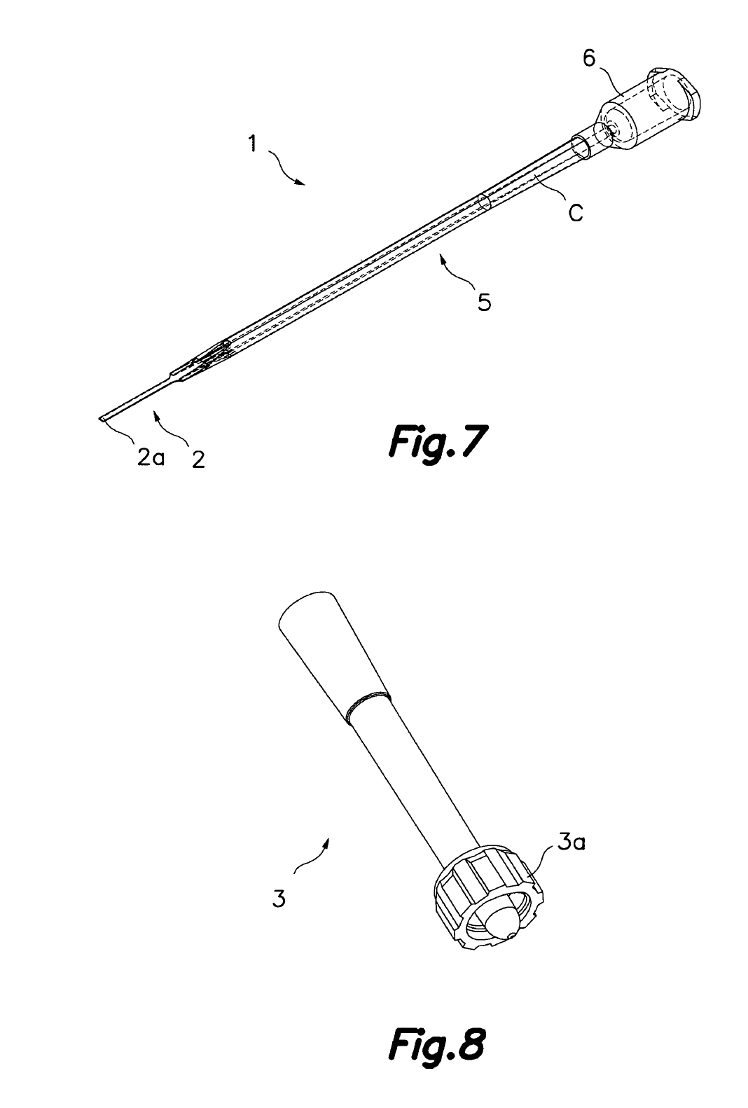

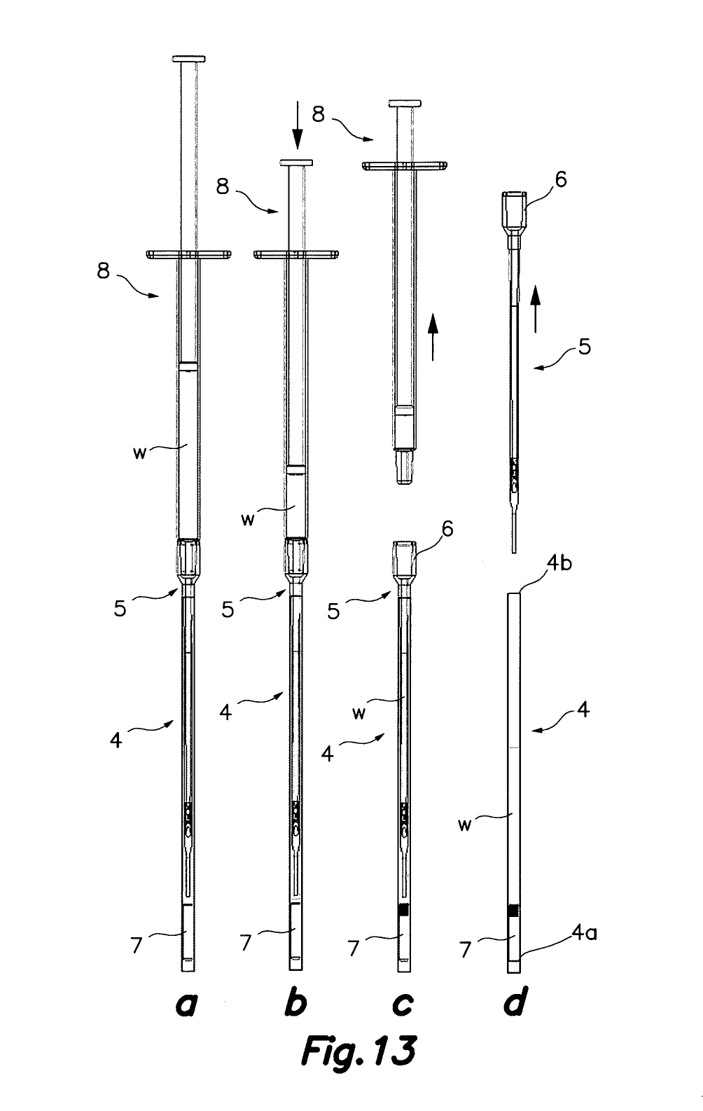

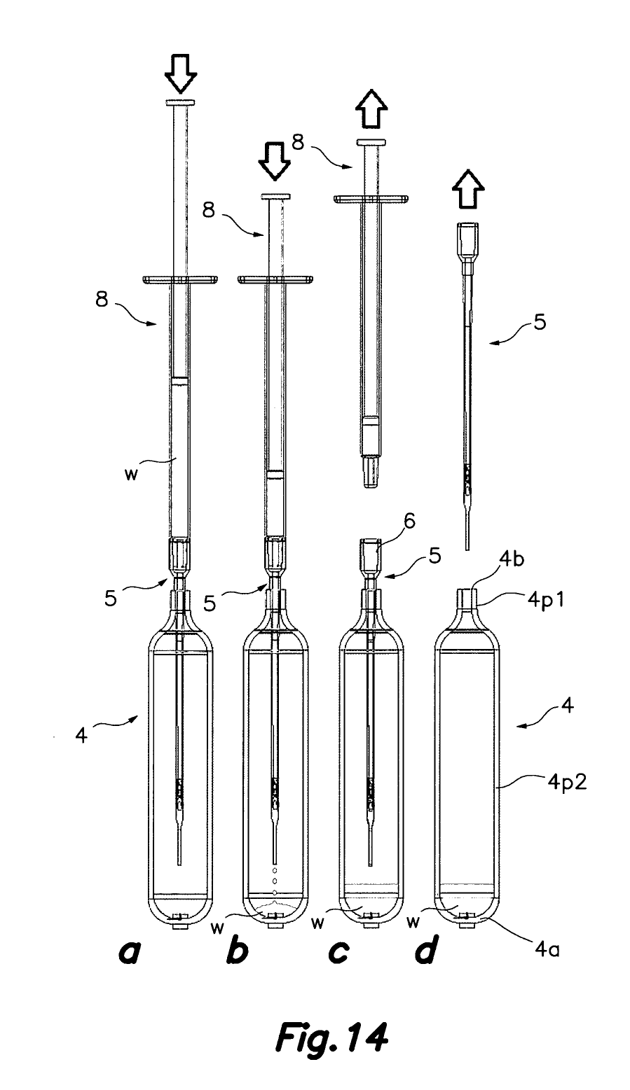

The device 1 provided by the first aspect of the invention comprises a handle portion 5, which for the embodiment illustrated in FIGS. 1 to 7, and 9 to 11, is an elongated body 5 which has an inner longitudinal channel C (indicated in FIGS. 1 to 3 and FIG. 7) defined through its whole length, arranged for conducting a warming solution (W in FIGS. 13 and 14) there through and for applying it over a biological material (not shown) while loaded on a loading portion 2a, for an in-situ warming.

As shown in FIGS. 1 to 7, 9 and 11, the loading portion 2a of the device 1 is, for the embodiment illustrated, a first end portion of an attachment support 2 arranged at a first end 5a of said elongated body 5.

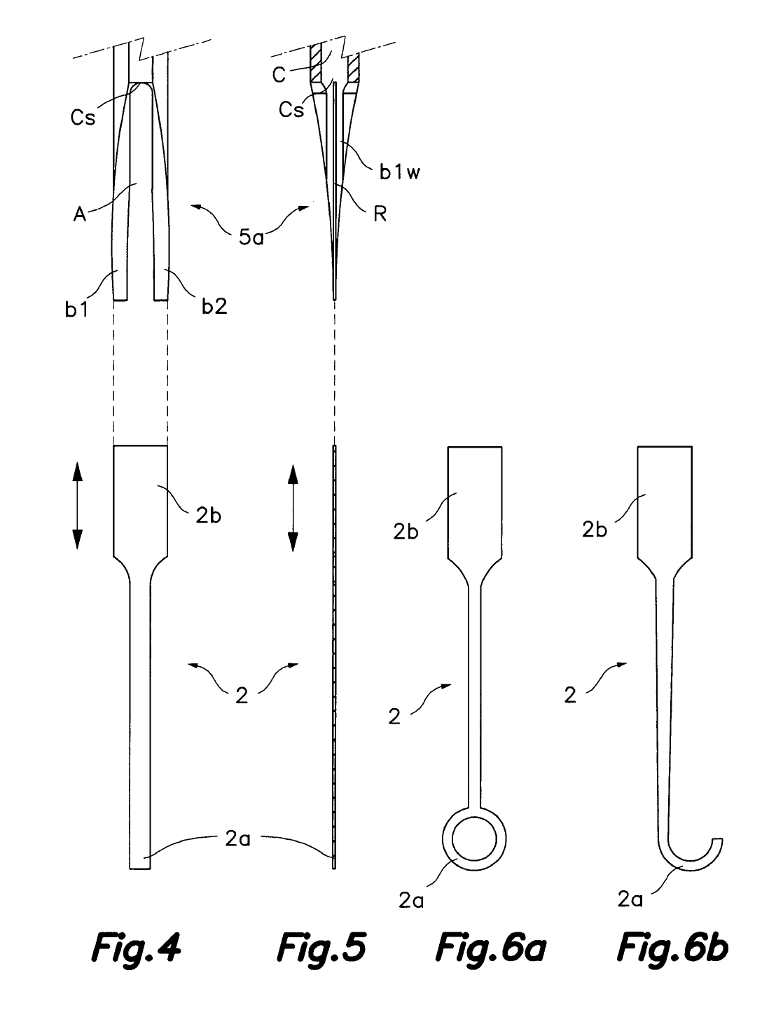

Three different embodiments for the attachment support 2 are shown in the appended Figures, one, illustrated in detail in FIGS. 4 and 5, for which the attachment support 2 is a thin planar film, another one, shown in FIG. 6a, for which the attachment support 2 has a tip 2a in the form of a ring, and a third one, shown in FIG. 6b, for which the attachment support 2 has a tip 2a in the form of a loop.

As can be seen particularly in FIGS. 1 to 3 and FIGS. 4 and 5, the inner channel C has an outlet Cs faced towards the attachment support 2, in order to direct the warming solution circulating there through towards the biological material loaded on the loading portion 2a.

Said attachment support 2 is, for the embodiment illustrated in FIGS. 4 and 5, removably coupled to said first end 5a of the elongated body 5, or elongated handle portion 5, by means of the introduction and displacement (as shown by arrows in FIGS. 4 and 5), in a guided manner, of side edges of a portion 2b of the attachment support 2 into faced U-shaped profile arms b1, b2 (see FIG. 4), decreasing in section towards their free ends, and into two respective slots R (only one illustrated in FIGS. 3 and 5) defined longitudinally in respective inner faced walls of the U-shaped profile arms b1, b2, only one of said inner faced walls having been illustrated, particularly the one indicated by the reference b1w in FIG. 5.

For the embodiment illustrated in FIGS. 4 and 5 the attachment support 2 is coupled to the elongated body 5, as explained in the above paragraph, covering part of the through opening A, shown at its maximum dimensions in FIG. 4, before the attachment support 2 is coupled to the elongated body 5, and at its lower dimensions in FIG. 2, once the attachment support 2 has been coupled thereto, without been introduced completely until the end of slots R.

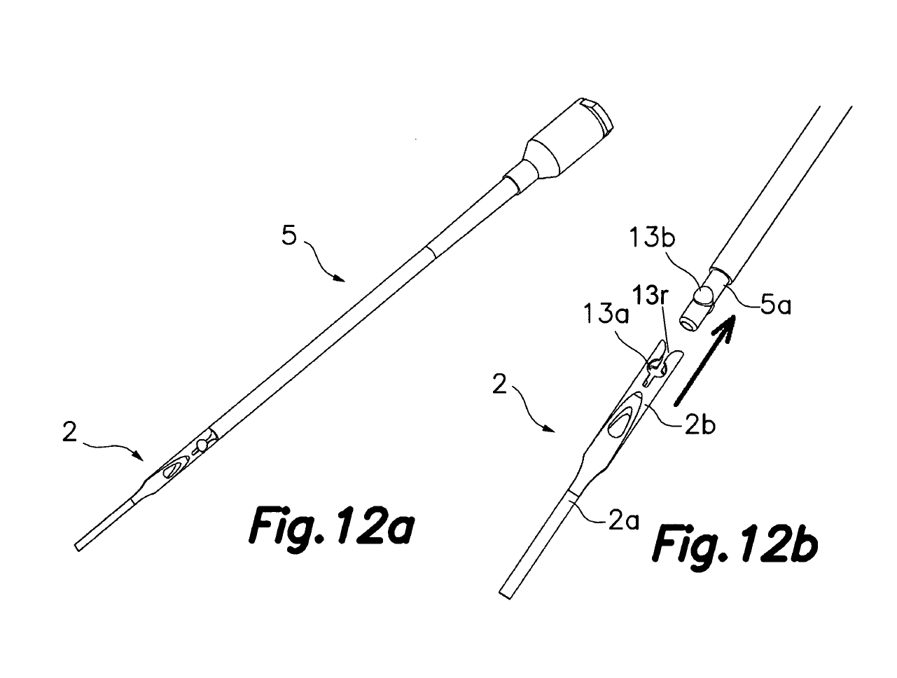

FIGS. 12a and 12b show an alternative embodiment to the one illustrated by FIGS. 4 and 5, for which the device 1 includes coupling means for the attachment support to the first end 5a of the elongated handle portion 5 different from the ones of FIGS. 4 and 5. Said coupling means comprise a coupling configuration at portion 2b of the attachment support 2 which is formed by a through hole 13a into which enter and fit respective protuberances 13b of a matching coupling configuration of the first end 5a of the elongated handle portion 5, said protuberances 13b entering said holes 13a through an access slot 13r, by elastically deforming the material of portion 2b which delimits said access slot 13r.

For the illustrated embodiment the biological material can be loaded on any of the two sides of the loading portion 2a, as, as shown in FIG. 5, the attachment support is arranged on the symmetry longitudinal axis of the channel C, i.e. at the centre of outlet Cs, and thus the warming solution (W in FIGS. 13 and 14) exiting through outlet Cs (see FIGS. 4 and 5) will flow along both of said two sides, and through the opening A illustrated in FIG. 2. However, for further embodiments (not shown), the biological material could also be loaded on one of the support's faces, so said support could be shifted from the symmetry longitudinal axis, being attached to the handle by either some slot-like method, or being part of the handle itself.

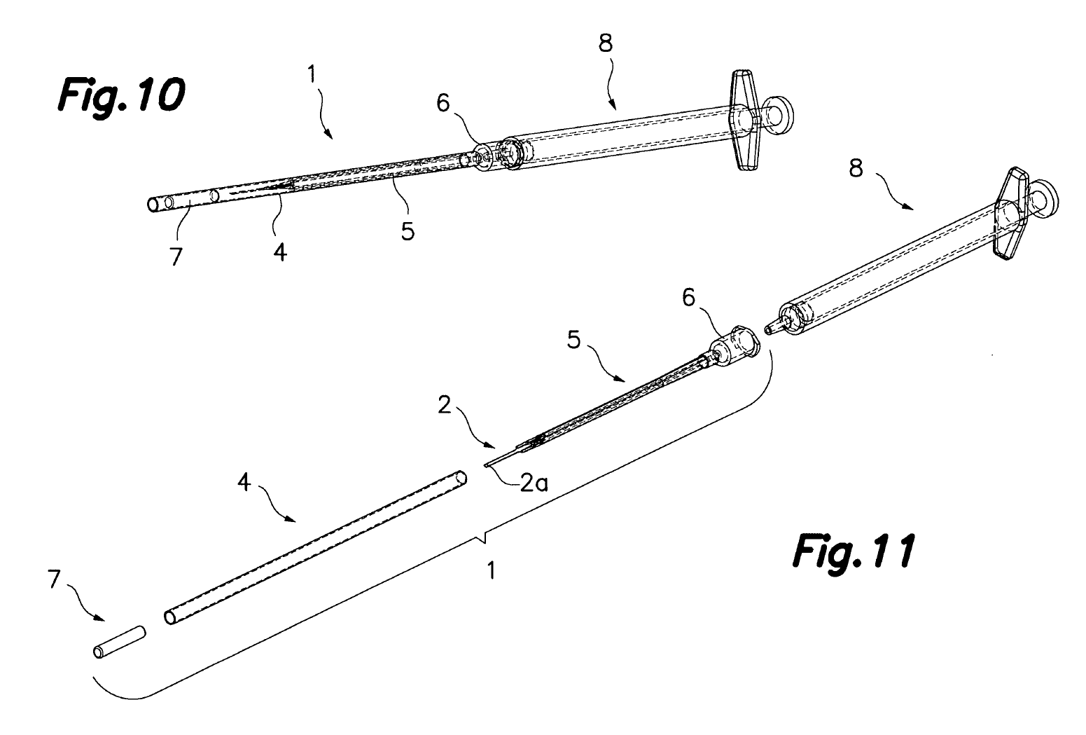

According to the embodiment illustrated by FIG. 10 and FIG. 11, the device 1 further comprises a cover straw 4, closed at an end by means of a cotton plunger 7, and coupled to the elongated body 5 as illustrated in FIG. 10, i.e. covering most of its length, thus protecting it from mechanical damage and also protecting the biological material loaded thereon.

As explained in a previous section, said cover straw 4 is dimensioned to receive and hold all the volume of the applied warming solution together with the biological material suspended there in, and is also provided for being inserted into an insemination catheter and for its use to transfer the biological material contained there in to the uterus of an animal.

As shown in FIGS. 1, 2 and 3, the inner channel C has an inlet Ci at a second end 5b of the elongated body 5, connected to an outlet of a warming solution source, by means of a connector 6.

For the embodiments illustrated in FIG. 10 and FIG. 11, said warming solution source is a syringe 8, which is used by introducing its tip into connector 6, and propelling its content into the inner channel C.

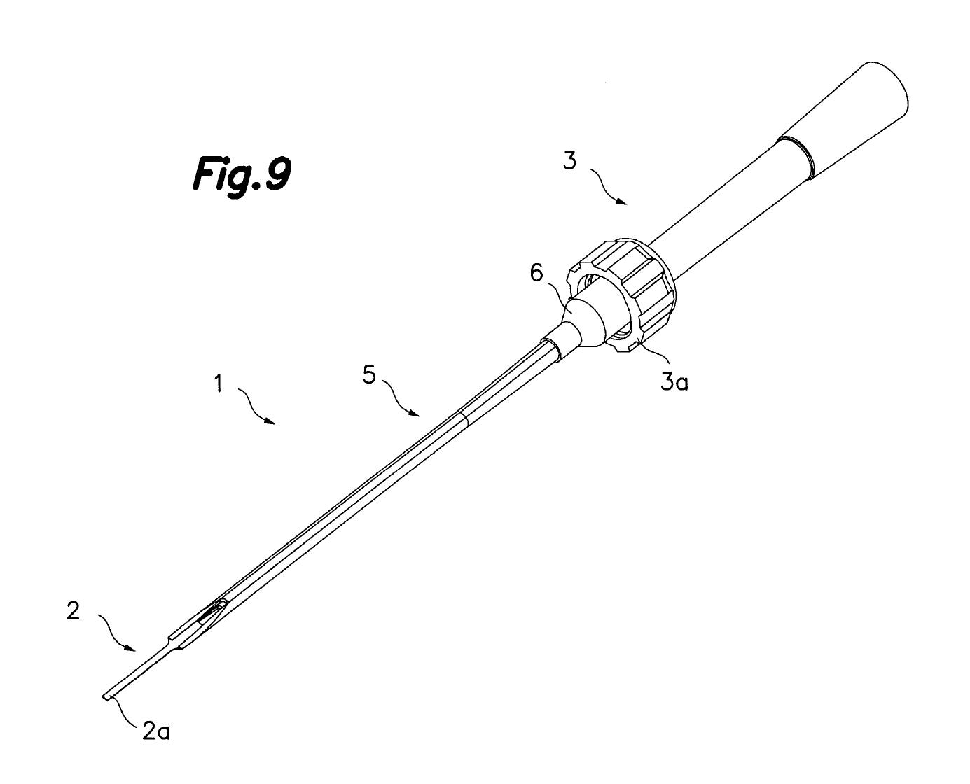

FIG. 9 illustrates another embodiment for which the warming solution source includes a LUER-Lock pipette 3, coupled to the connector 6.

FIG. 13 shows the device of the first aspect of the invention for the above mentioned embodiment for which the container 4 is a semen straw, and the device is being used to transfer biological material (not shown) to the interior of said semen straw 4 with a syringe 8.

Particularly at view a of FIG. 13 the output port of said syringe 8 is coupled to the input connector 6 of the elongated handle portion 5 and the semen straw 4 is coupled to the handle portion 5 by the introduction of the latter through the straw mouth 4b, said coupling not allowing air to escape from the cover straw 4 interior via said mouth 4b.

Following the sequence of images of FIG. 13, at view b the plunger of the syringe 8 is being pushed such that its contents, i.e. warming solution W together with the swept biological material (not shown), enters the cover straw 4 and pushes inner air (not shown) to make it escape out of the straw 4 through porous plug 7, thus occupying its previous place.

At view c of FIG. 13 the syringe 8 is uncoupled and withdrawn from the elongated handle portion 5 and at view d the elongated handle portion 5 is retrieved from the cover straw 4, the warming solution W with the biological material suspended therein remaining into the cover straw 4 as no air counter pressure exists which could push it there out.

FIG. 14 shows an alternative embodiment for which, instead of a cover straw, the container 4 is a small bottle 4 having two portions with different section dimensions: a first one 4p1 including the container mouth 4b and a second one 4p2 with a wider section dimension.

In this case, the introduction of the warming solution W into the bottle 4 is performed also from a syringe 8, following the sequence of views a to d of FIG. 14.

At view b the plunger of the syringe 8 is being pushed such that its contents, i.e. warming solution W together with the swept biological material (not shown), enters the bottle 4 by dripping (see view b) and settles in the bottom of the second portion 4p2 (see views b, c and d), and remains there even if a slight inner air counter pressure is created, as the latter would not ever push the settled warming solution W towards the container mouth 4b, due to the dimensional ratio of the sections of portions 4p1 and 4p2.

At view c of FIG. 14 the syringe 8 is uncoupled and withdrawn from the elongated handle portion 5 and at view d the elongated handle portion 5 is retrieved from the bottle 4, the warming solution W with the biological material suspended therein remaining onto the bottom of bottle 4, as no air counter pressure is pushing it out towards the mouth 4b.

Next a study carried out by the present inventors, in order to test the goodness of the device and the use provided by the present invention, is disclosed.

### In Vitro Embryo Production

Ovaries were collected from slaughtered prepubertal calves and cows and transported to the laboratory in physiological saline at approximately 37° C. within 1-2 h. Oocytes were obtained by aspiration of follicles using a 20 g needle fitted with a 5 mL syringe. Follicular oocytes surrounded by at least two layers of granulose cells and with an evenly granulated cytoplasm were selected for in vitro maturation (IVM). The medium used for maturation was TCM-199 supplemented with 10% (v/v) fetal calf serum (FCS), 10 ng mL-1 epidermal growth factor and 50 μg mL-1 gentamicin. The oocytes were incubated in 500 μL of medium in 4-well plates in a humidified 5% CO2 air atmosphere at 38.5° C. for 24 h.

Following maturation, the COCs were washed four times in PBS and then in the fertilization medium before being transferred, in groups of up to 50, to 4-well plates containing 250 μl of fertilization medium per well (Tyrode's medium with 25 mM sodium bicarbonate, 22 mM Na-lactate, 1 mM Na-pyruvate and 6 mg mL-1 fatty acid-free BSA and 10 mg mL-1 heparin-sodium salt). Motile spermatozoa were obtained by centrifuging frozen-thawed sperm from Asturian bulls on a discontinuous Percoll density gradient (2.5 mL 45% (v/v) Percoll over 2.5 mL 90% (v/v) Percoll) for 8 min at 700 g at room temperature. The pellet was washed in Hepes-buffered Tyrode's medium and pelleted again by centrifugation at 100 g for 5 min. Fertilization was conducted in 500 μL of fertilization medium with a final concentration of 1×106 spermatozoa mL-1 for 22 h at 38.5° C. in a 5% CO2 humidified air atmosphere. Variation between individual bulls was avoided by mixing equal sperm samples from two bulls in all the experiments.

After co-culture with spermatozoa for 22 h, presumptive zygotes were transferred to 25 μl culture droplets (1 embryo/μl) under mineral oil. The embryos were incubated in synthetic oviductal fluid medium for 7 or 8 days at 38.5° C. in a 5% CO2, 5% O2 humidified atmosphere. Cleavage rates were recorded at 48 h post-insemination and numbers of blastocysts were determined on post-insemination days 7 and 8.

### Embryo Vitrification

The holding medium (HM) used to formulate the vitrification-warming solutions was TCM 199 Hepes buffered with 20% FCS. All steps were performed under a laminar flow hood heated at 38.5° C. using a stereomicroscope to visualize each step. Blastocysts were transferred into equilibration solution (ES) containing 7.5% ethylene glycol (EG) and 7.5% dimethyl sulphoxide (DMSO) in HM for 10 to 15 min. After an initial shrinkage, blastocysts regained their original volume and they were then moved to the vitrification solution (VS) composed 15% EG, 15% DMSO and 0.5 M sucrose dissolved in HM. After incubation for 60 sec, one blastocyst was loaded onto the embryo attachment tip 2a, the remaining solution was almost removed to leave only a thin layer covering the blastocyst and the sample was quickly immersed into liquid nitrogen. Subsequently, the plastic cap, or cover straw 4, was pulled over the device 1. The entire process from immersion in VS to plunging into liquid nitrogen was completed within 60-90 sec. The loaded devices 1 were stored in liquid nitrogen at −196° C.

### Embryo Warming and Transfer (in Field Conditions)

Immediately after removing the whole device 1 from the liquid nitrogen, 100 microliters of warming solution (1 M sucrose dissolved in HM) were added directly to the protective cap 4 through the inner channel C of hollow handle 5 using a standard syringe 8. After 1 min, 100 microliters of HM were added to the protective cap 4, in the same manner. Cryoprotectant diffusion out of the embryo was carried out by gentle movement of the protective cap 4. Uterine transfer of the embryos was performed immediately after dilution using the same procedure than an artificial insemination.

### Prototypes Testing Results

In preliminary experiments carried out in the laboratory of the present inventors, no embryos were lost when warming solutions were added through the device 1 provided by the present invention, so that all the embryos remained in the cover straw 4 after warming.

A person skilled in the art could introduce changes and modifications in the embodiments described without departing from the scope of the invention as it is defined in the attached claims.

### REFERENCES

Abdelhafez F F, Desai N, Abou-Setta A M, Falcone T, Goldfarb J. (2010) Slow freezing, vitrification and ultra-rapid freezing of human embryos: a systematic review and meta-analysis. Reprod Biomed Online 20:209-222.
Antinori M, Licata E, Dani G, Cerusico F, Versaci C, Antinori S (2007) Cryotop vitrification of human oocytes results in high survival rate and healthy deliveries. Reprod Biomed Online 14, 72-9.
Bogliolo L, Ariu F, Rosati I, Zedda M T, Pau S, Naitana S (2006) Vitrification of immature and in vitro matured horse oocytes. Reprod Fertil Develop. 18, 149-150 [Abstract].
Cobo A, Kuwayama M, Perez S, Ruiz A, Pellicer A, Remohi J (2007) Comparison of concomitant outcome achieved with fresh and cryopreserved donor oocytes vitrified by the Cryotop method. Fertil Steril.
Chen S U, Lien Y R, Cheng Y U, Chen H F, Ho H N, Yang Y S (2001) Vitrification of mouse oocytes using closed pulled straws (CPS) achieves a high survival and preserves good patterns of meiotic spindles, compared with conventional straws, open pulled straws (OPS) and grids. Human Reproduction 16, 2350-2356.
Chian R C, Kuwayama M, Tan L, Tan J, Kato O, Nagai T (2004) High survival rate of bovine oocytes matured in vitro following vitrification. J Reprod Dev 50, 685-96.
Du Y, Zhang Y, et al. (2007) Simplified cryopreservation of porcine cloned blastocysts. Cryobiology 54, 181-7.
Gasparrini B, Attanasio L, De Rosa A, Monaco E, Di Palo R, Campanile G (2007) Cryopreservation of in vitro matured buffalo (Bubalus bubalis) oocytes by minimum volumes vitrification methods. Anim Reprod Sci 98, 335-42.
Hochi S, Terao T, Kamei M, Kato M, Hirabayashi M, Hirao M (2004) Successful vitrification of pronuclear-stage rabbit zygotes by minimum volume cooling procedure. Theriogenology 61, 267-75.
Isachenko V, Alabart J L, et al. (2003a) New technology for vitrification and field (microscope-free) warming and transfer of small ruminant embryos. Theriogenology 59, 1209-18.
Isachenko V, Selman H, Isachenko E, Montag M, El-Danasouri I, Nawroth F (2003b) Modified vitrification of human pronuclear oocytes: efficacy and effect on ultrastructure. Reprod Biomed Online 7, 211-6.
Isachenko V V, Isachenko E F, Ostashko F I, Grishchenko V I (1997) Ultrarapid freezing of rat embryos with rapid dilution of permeable cryoprotectants. Cryobiology 34, 157-64.
Iwayama H, Hochi S, Kato M, Hirabayashi M, Kuwayama M, Ishikawa H, Ohsumi S, Fukui Y (2004) Effects of cryodevice type and donors' sexual maturity on vitrification of minke whale (Balaenoptera bonaerensis) oocytes at germinal vesicle stage. Zygote 12, 333-8.
Kobayashi K, Nagashima H, Yamakawa H, Kato Y, Ogawa S (1990) The survival of whole and bisected rabbit morulae after cryopreservation by the vitrification method. Theriogenology 33, 777-88.
Kuwayama M, Vajta G, Kato O, Leibo S P (2005) Highly efficient vitrification method for cryopreservation of human oocytes. Reprod Biomed Online 11, 300-8.
Lane M, Schoolcraft W B, Gardner D K (1999) Vitrification of mouse and human blastocysts using a novel cryoloop container-less technique. Fertil Steril 72, 1073-8.
Laowtammathron C, Lorthongpanich C, Ketudat-Cairns M, Hochi S, Parnpai R (2005) Factors affecting cryosurvival of nuclear-transferred bovine and swamp buffalo blastocysts: effects of hatching stage, linoleic acid-albumin in IVC medium and Ficoll supplementation to vitrification solution. Theriogenology 64, 1185-96.
Leibo S P, Mapletoft R J (1998) Direct transfer of cryopreserved cattle embryos in North America. Proc. 17th Annual Convention of the American Embryo Transfer Association (AETA), October, San Antonio, Tex., 91-98.
Liebermann J, Tucker M J, Graham J R, Han T, Davis A, Levy M J (2002) Blastocyst development after vitrification of multipronuclear zygotes using the Flexipet denuding pipette. Reprod Biomed Online 4, 146-50.
Li T, Mai Q, Gao J, Zhou C. (2010) Cryopreservation of human embryonic stem cells with a new bulk vitrification method. Biol Reprod 82(5):848-853).
Lucena E, Bernal D P, Lucena C, Rojas A, Moran A, Lucena A (2006) Successful ongoing pregnancies after vitrification of oocytes. Fertil Steril 85, 108-11.
Martino A, Songsasen N, Leibo S P (1996) Development into blastocysts of bovine oocytes cryopreserved by ultra-rapid cooling. Biol Reprod 54, 1059-69.
Matsumoto H, Jiang J Y, Tanaka T, Sasada H, Sato E (2001) Vitrification of large quantities of immature bovine oocytes using nylon mesh. Cryobiology 42, 139-44.
Richards M, Fong C Y, Tan S, Chan W K, Bongso A. (2004) An efficient and safe xeno-free cryopreservation method for the storage of human embryonic stem cells. Stem Cells 22:779-789;
Stehlik E, Stehlik J, Katayama K P, Kuwayama M, Jambor V, Brohammer R, Kato O (2005) Vitrification demonstrates significant improvement versus slow freezing of human blastocysts. Reprod Biomed Online 11, 53-7.
Succu S, Leoni G G, Bebbere D, Berlinguer F, Mossa F, Bogliolo L, Madeddu M, Ledda S, Naitana S (2007) Vitrification devices affect structural and molecular status of in vitro matured ovine oocytes. Mol Reprod Dev 74, 1337-44.
Szell A, Shelton J N (1986) Sucrose dilution of glycerol from mouse embryos frozen rapidly in liquid nitrogen vapour. J Reprod Fertil 76, 401-8.
Ushijima H, Yoshioka H, Esaki R, Takahashi K, Kuwayama M, Nakane T, Nagashima H (2004) Improved survival of vitrified in vivo-derived porcine embryos. J Reprod Dev 50, 481-6.
Vajta G, Holm P, Kuwayama M, Booth P J, Jacobsen H, Greve T, Callesen H (1998) Open Pulled Straw (OPS) vitrification: a new way to reduce cryoinjuries of bovine ova and embryos. Mol Reprod Dev 51, 53-8.
Wobus A M. (2001) Potential of embryonic stem cells. Mol Aspects Med 22:149-164).

1. A device for manipulating biological materials in a process of cryopreservation, having a loading portion for loading biological material thereon comprising:
an elongated handle portion with a channel defined through at least part of its interior, along its length, said channel being arranged for conducting a warming solution there through and for applying it over said biological material while loaded on said loading portion, for an in-situ warming; and
a container closed at an end and having a mouth, said container being arranged, dimensioned and configured for:
being removably coupled to said elongated handle portion by the introduction of the latter through said container mouth, covering at least said loading portion, such that, when coupled, inner air cannot escape via said mouth; and
receiving, when coupled, both said applied warming solution and said biological material, once warmed and swept away from said loading portion by said applied warming solution, without inner air pushing said warming solution towards the container mouth by an inner air counter pressure.

2. A device as per claim 1, wherein said container is a cover straw which is closed at said end by a plug made of a porous material permeable to gases and impermeable to liquids, such that, when said warming solution is being introduced into the container inner air can escape through said plug to allow the warming solution to occupy inner air previous place, in order not to generate said inner air counter pressure.

3. A device as per claim 2, wherein said plug is also made of a second material that once wet by said warming solution does not let liquids percolate there through and thus becomes a plunger which, by sliding, can propel the container contents through said mouth when the container is uncoupled from said elongated handle portion and said plunger is pushed by external actuation means.

4. A device as per claim 3, wherein said cover straw is a semen straw.

5. A device as per claim 1, wherein said container is a cover straw made of a porous material, such that, when said warming solution is being introduced into the container inner air can escape through the cover straw walls to allow the warming solution to occupy inner air previous place, in order not to generate said inner air counter pressure.

6. A device as per claim 1, wherein said container has two portions with different section dimensions, a first one including said mouth and a second one with a wider section dimension, the dimensional ratio between said two portions section dimensions providing that said warming solution enters the container by dripping, settles in the second portion bottom and is not pushed towards the container mouth by inner air counter pressure, not even when retrieving said elongated handle portion.

7. A device according to claim 1, wherein said loading portion is part of an attachment support arranged at a first end of said elongated handle portion, said inner channel having an outlet faced towards said attachment support.

8. A device according to claim 7, wherein said inner channel has an inlet at a second end of said elongated handle portion.

9. A device according to claim 8, wherein said second end of said elongated handle portion comprises a connector for connecting the channel inlet to an outlet of a warming solution source.

10. Use of the device of claim 1 for manipulating biological materials in a process of cryopreservation, comprising simultaneously warming and transferring a cryopreserved biological material loaded on a device loading portion, by conducting and applying a warming solution on said biological material loaded on said device loading portion.

11. Use as per claim 10, comprising performing said warming solution conduction through an inner channel of said device.

12. Use as per claim 10, comprising performing said transfer from a handle portion of said device defining said inner channel to a container of said device.

13. Use as per claim 12, comprising performing a second transfer from said container to a final target.

14. Use as per claim 10, wherein said biological material is a biological material, or a sample thereof, selected from the group comprising: an oocyte, an embryo, a cell and a tissue.

15. Use as per claim 10, wherein said cryopreserving is carried out by a vitrification technique.

16. (canceled)
